(19) United States
(12) Patent Application Publication
(10) Pub. No.: US 2005/0187426 A1
(43) Pub. Date: Aug. 25, 2005

(54) System and method for synchronizing the heart rate variability cycle with the breathing cycle

(76) Inventor: Stephen Bennett Elliott, Allen, TX (US)

Correspondence Address: Stephen Bennett Elliott, 702 Buffalo Springs Drive, Allen, TX, 75013, US

(21) Appl. No.: 10/784,266

(22) Filed: Feb. 24, 2004

(51) Int. Cl. A61B 5/04; A61M 21/00

(52) U.S. Cl. 600/26; 128/905

(57) ABSTRACT

The invention consists of a method and system for achieving coherence of heart rate variability by synchronizing the heart rate variability cycle to the breathing cycle and by consciously synchronizing the breathing cycle with an external reference that is closely aligned with the frequency of the natural heart rate variability cycle. Various means of representing the cycle of the external reference are provided including visual, audible, and sensory indicators. An instructive method is provided that teaches the subject to consciously synchronize their inhalation with the positive going aspect of the external reference cycle and their exhalation with the negative going aspect of the external reference cycle.

### FIELD OF THE INVENTION

The present invention relates to human physiology, and in particular to a method and system for allowing a human subject to consciously control physiological processes, more particularly, it allows a human subject to achieve synchronization of the natural cycle of heart rate with the breathing cycle.

### BACKGROUND OF THE INVENTION

The human heart is known to have its own nervous system and its own natural tendency toward rhythm. For purposes of this invention, there are two primary aspects to this rhythm, the heartbeat rate, and the rate at which the heartbeat rate changes otherwise known as heart rate variability. Heartbeat rate is usually specified in absolute number of heartbeats occurring during a specified period. Heartbeat rate variability, otherwise know as heart rate variability is the change in heartbeat rate as occurs during a specified period. Henceforth, heartbeat rate variability will be referred to as heart rate variability.

While the heart has its own tendency toward rhythm, it is closely coupled to breathing. The relationship is such that as inhalation occurs, the heartbeat rate tends to increase and as exhalation occurs, the heartbeat rate tends to decrease. It is important to note that while the heartbeat rate and breathing rate influence each other, the relationship is a plesiochronous one, that is, they are independent rhythms that strongly influence but do not directly control each other.

It is generally recognized that heart rate variability is an indicator of physiological and emotional state, that is, irregular incoherent heart rate variability indicates a condition of physiological/psychological stress. Alternatively, a highly regular coherent heart rate variability is indicative of a condition of physiological/psychological harmony.

Accordingly, it is highly desirable to achieve and maintain a highly coherent heart rate variability as life circumstances permit. This having been said, with proper training and the application of the present invention, it is possible for a human subject to rapidly achieve the desired state of high coherence of heart rate variability and to reinforce that coherence on an ongoing basis.

The present invention takes advantage of the relationship between the breathing cycle and the natural heart rate variability cycle to bring heart rate variability to the desired state of coherence and the human subject to the resultant state of physiological and emotional harmony. It accomplishes this via synchronization of the heart rate variability cycle with the breathing cycle.

### SUMMARY OF THE INVENTION

As previously described, a relationship exists between the heartbeat rate specified in terms of heart rate variability, and the breathing cycle. While the heart has its own tendency toward a natural variable rhythm, there is a strong correlation with breathing according to this specific relationship: as inhalation occurs, there is a tendency for the heartbeat rate to increase, as exhalation occurs, there is a tendency for the heartbeat rate to decrease. In a relaxed human subject, the effect of the breathing cycle on the heart rate variability cycle is extremely strong. In fact, the heart rate variability cycle will synchronize with the breathing cycle if the breathing cycle is highly attuned to the periodicity of the natural heart rate variability cycle.

The present invention accomplishes this by presenting the human subject with an accurate external timing reference to which the breathing can be consciously synchronized. This external timing reference is centered about the average heart rate variability cycle of 0.085 Hertz or a period of 11.7 seconds. When the breathing is consciously synchronized to this external reference signal, the heart rate variability cycle will synchronize with it. Once the heart rate variability cycle synchronizes with the breathing cycle, they remain synchronized as long as the breathing cycle remains highly aligned with the external timing source. In this way, the human subject can remain in the desired state of coherence of heart rate variability for extended periods of time. Ultimately, this builds familiarity with the desired psycho-physiological condition such that the state can be realized at will with or without the external timing reference.

For purposes of the present invention, we can consider the cycles of heart rate variability, the periodicity of increasing and decreasing of heartbeat rate, and the breathing cycle, the periodicity of inhalation and exhalation, to be two independent cycles. The relative synchronization of these cycles can vary between 0 and 180 degrees. When these cycles are completely out of phase, heart rate variability is maximally incoherent, when these cycles are completely in phase heart rate variability is maximally coherent

### BRIEF DESCRIPTION OF THE DRAWING FIGURES

The accompanying drawing figures incorporated in and forming a part of this specification illustrate several aspects of the invention, and together with the description serve to explain the principles of the invention.

FIG. 1 depicts the relative relationship between the heart rate variability cycle and breathing cycle.

FIG. 2 depicts the natural heart rate variability cycle and breathing cycle moving from misalignment to alignment and resultant heart rate variability pattern.

FIG. 3 depicts the block diagram of the preferred embodiment of the present invention.

FIG. 4 presents a table defining frequency programming options, resulting periods, and timing generator output rates.

FIG. 5 depicts the changing of the visual array at 9 distinct intervals.

FIG. 6 depicts the invention as a software application running on a computer providing visual, audible, and sensory indicators.

### DETAILED DESCRIPTION OF THE PREFERRED EMBODIMENTS

The embodiments set forth below represent the necessary information to enable those skilled in the art to practice the invention and illustrate the best mode of practicing the invention. Upon reading the following description in light of the accompanying drawing figures, those skilled in the art will understand the concepts of the invention and will recognize applications of these concepts not particularly addressed herein. It should be understood that these concepts and applications fall within the scope of the disclosure and the accompanying claims.

The present invention allows a human subject to achieve coherence of heart rate variability by synchronizing the heart rate variability cycle with the breathing cycle. This is accomplished by providing an external timing reference in the form of an audible, visual, or sensory signal, indicating when the subject should begin inhalation, when the subject should end inhalation, when the subject should begin exhalation, and when the subject should end exhalation. This is repeated in a cyclic fashion, inhalation leading to exhalation, exhalation leading to inhalation, and so forth. The external reference presents a signal to the human subject centered around 0.085 Hertz for a period of 11.8 seconds, the heart rate variability center frequency of the typical human. When the typical human subject breathes at this rate the heart rate variability cycle will synchronize with the breathing cycle, thereby maximizing the coherence of the heart rate variability cycle.

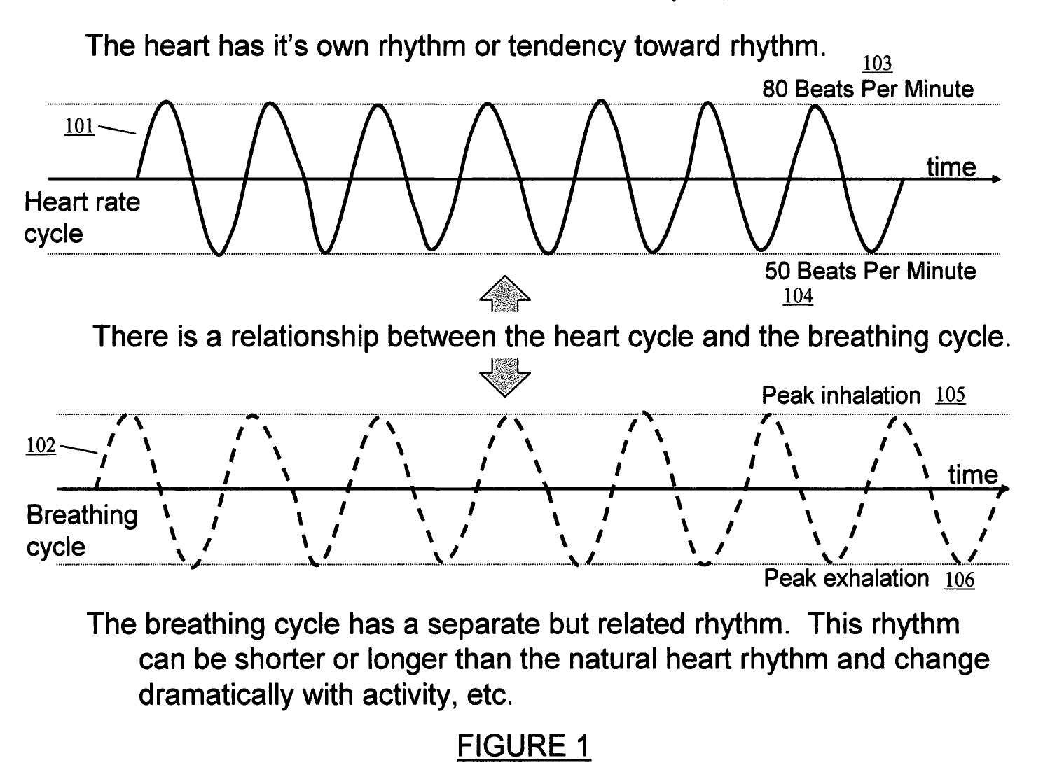

With reference to FIG. 1, the heart has its own nervous system and a tendency toward its own natural rhythm. For the purposes of discussion, FIG. 1 defines the peak positive rhythm as 80 beats per minute (BPM) 103 and the peak negative rhythm as 50 beats per minute (BPM) 104. Let it be clear that 80 beats per minute as the positive peak and 50 beats per minute as the negative peak are merely used for purposes of example. The breath is under control of the human central nervous system and operates with a largely independent rhythm. Yet, there is a strong correlation between the breath cycle 102 and the natural heart rate variability cycle 101 as described prior.

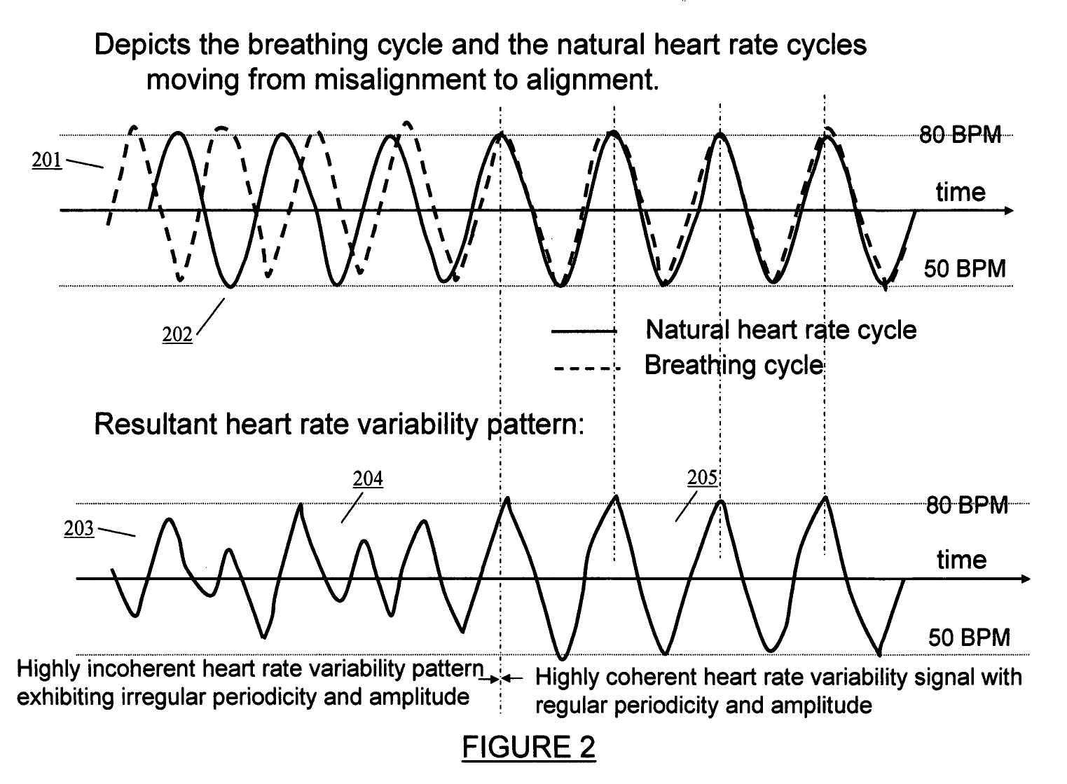

With reference to FIG. 2, synchrony between the natural heart rate variability cycle 202, and the cycle of breathing 201, is highly variable ranging from being highly synchronous (in-phase) to being highly asynchronous (out of phase). This results in a highly periodic and coherent heart rate variability pattern 205 vs. a highly aperiodic and incoherent heart rate variability pattern 204, respectively. The primary application of the present invention is to lead a human subject to the preferred state of highly periodic and coherent heart rate variability, 205, both in time and amplitude.

Before entering into the proceeding description of the preferred embodiment of the present invention, it is assumed that the invention may be packaged in numerous ways and or incorporated into numerous alternative packaging configurations ranging from fountain pen, to wristwatch, to cell phone sized instruments, to the like representation on personal computer, television set, or like displays. Those skilled in the art will understand the concepts of the invention and will recognize applications of these concepts not particularly addressed herein. It should be understood that these concepts and applications fall within the scope of the disclosure and the accompanying claims.

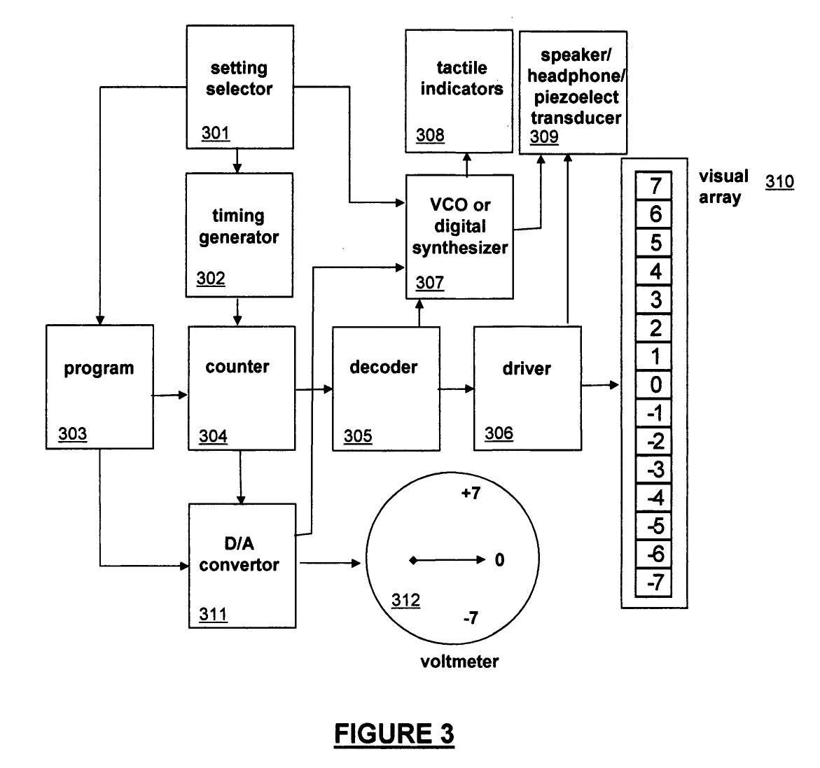

With reference now to FIG. 3, the logical system is quite simple consisting fundamentally of a programmable timing generator and alternative presentation mechanisms including audible, visual, and sensory indicators.

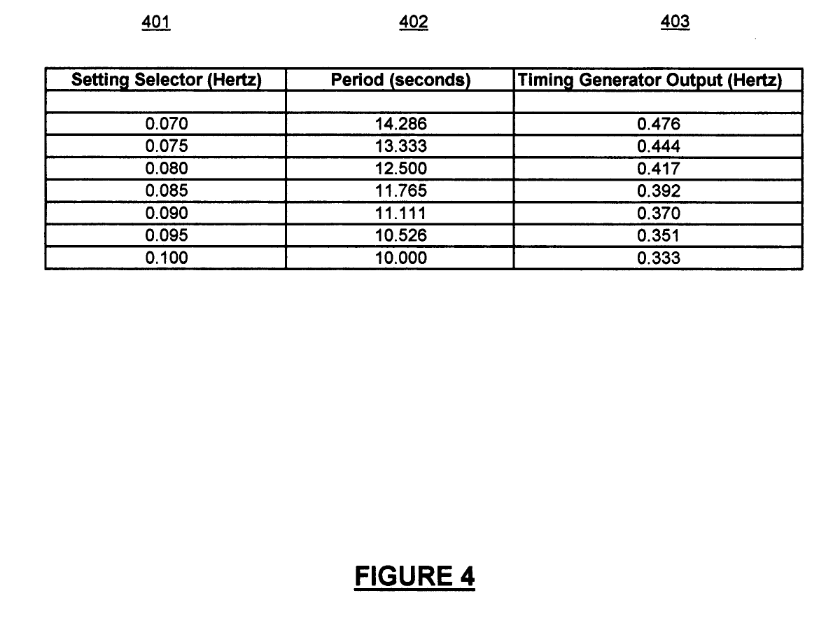

A detailed discussion of the logical system will now ensue. Settings Selector 301 provides the user with the ability to select the precise frequency of the breathing cycle centered around 0.085 Hertz. Steps are provided in 0.005 Hertz steps on either side of the center frequency to accommodate for age and personal comfort. In other words, frequency steps are provided at 0.080 Hertz, 0.075 Hertz, and 0.070 Hertz on the low end, and at 0.090 Hertz, 0.095 Hertz, and 0.100 Hertz on the high end. These steps are depicted in the table of FIG. 4, column 401.

Settings Selector 301 also provides selection of alternative presentation methods including visual, audible, and sensory indicators. FIG. 3 depicts multiple indication methods including a visual array, as might exist of light emitting diodes, an analog voltmeter, an audio indication, and a sensory indication. These indications may be provided individually or in combination depending on the needs and desires of the user.

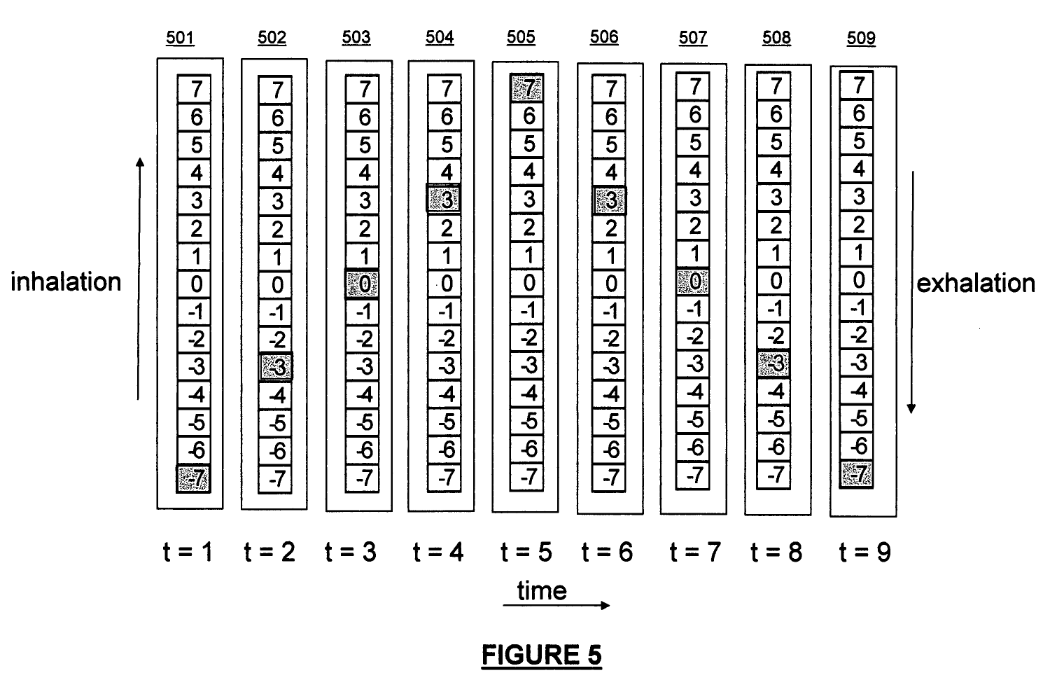

Timing Generator 302 provides necessary clock signals to Counter 304 under control of Setting Selector 301 such that visual, audible, and sensory indicators can be generated. This requires clock signals to be output to Counter 304 of varying frequency as is indicated in the third column of FIG. 4, Timing Generator Output 403. For convenience of this description, visual indication is delineated into fifteen (15) steps as is indicated by Visual Array 310. Consequently, a complete breath cycle consists of thirty (30) steps as is depicted in FIG. 5. FIG. 5 presents the status of the visual indicator at nine (9) points in time, shading indicating “activation” of individual array elements. In this case, a complete inhalation and exhalation is represented by the activation of the bottom most indicator, depiction by 501, to the top most indicator, depiction 505, and back again, depiction 509, the upward transition representing the period of inhalation and the downward transition representing period of exhalation. Consequently, inhalation ends and exhalation begins at the uppermost indicator, depiction 505, and exhalation ends and inhalation begins at the bottommost indicator, depictions 501 and 509. It should be noted that this is a logical convention as upward and downward transitions are typically of equal time. Note that for convenience, FIG. 5 depicts activation every 34 steps. Referring for a moment to FIG. 4, depending on Setting Selector settings, column 401, each individual indicator of FIG. 5 is illuminated for a period of time consistent with Timing Generator Output, column 403, ranging between the 0.33 seconds and 0.47 seconds, representing 0.100 Hertz and 0.070 Hertz respectively. In this way, a complete cycle of the visual display occurs in the specified period.

Counter 304 simply counts pulses from Timing Generator 302 in an up/down counter fashion from 0 to 15 and back to 0 under control of Program function 303. The output of Counter 304 is presented to Decoder 305 where it is decoded into 1 of 15 outputs. The outputs of Decoder 305, each associated with an individual element of Visual Array 310 are presented to Driver 306 which buffers and drives the visual elements. Voltage Controlled Oscillator function 307 converts the voltage output of Digital to Analog Convertor 311 to oscillations of varying frequency so as to alternatively provide audible and sensory representations of the cycle. Alternatively, function 307 may be a Digital Synthesizer, converting the digital output of Decoder 305 into analog outputs to drive Speakers, Headphones, and Sensory Indicators functions 308 and 309. Here a consistent convention is employed between visual, audible, and sensory indicators such that the inhalation phase is indicated by increasing frequency and the exhalation phase is indicated by decreasing frequency as would be experienced by a subject using either headphones or a vibrator. Consequently, the positive visual peak corresponds to positive audible peak frequency and positive sensory peak frequency. Similarly, the negative visual peak corresponds to negative audible peak frequency and negative sensory peak frequency. Therefore, the visual positive peak indication and audible and sensory positive peak indications relate directly to the positive peak heart rate, and, the negative visual peak indication and audible and sensory negative peak indications relate directly to the negative peak heart rate. Differing frequencies at which the speaker, headphones, and a vibrator may operate, are accounted for within the Voltage Controlled Oscillator/Digital Synthesizer function 307. Additionally, the output of Driver 306 is presented to the input of Speaker, Headphone, and Piezoelectric Transducer 309 for purposes of alternatively generating an audible indication when to end the inhalation phase and begin the inhalation phase, and, when to end the exhalation phase and begin the inhalation phase, as might be best characterized as a piezoelectric “chirp”. Selection of visual, audible, and sensory indicators occurs under control of Setting Selector 301.

The output of Counter 304 is presented to D/A Convertor 311 where it is converted to an analog signal for driving Voltage Controlled Oscillator/Digital Synthesizer 307 and alternatively analog Voltmeter 312.

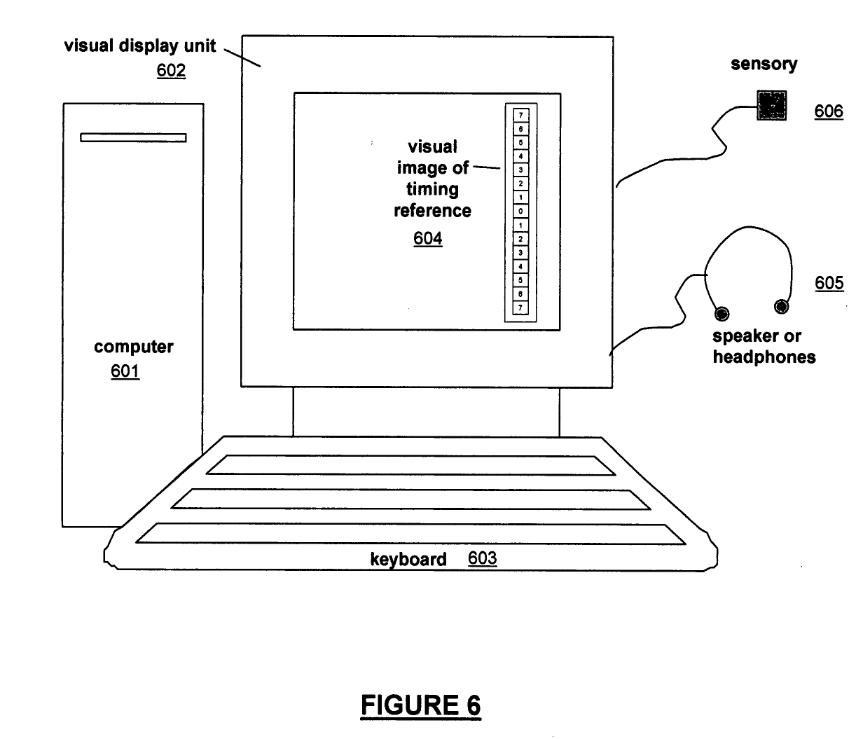

Those skilled in the art will recognize that the foregoing discussion describes the logic associated with the preferred embodiment of the present invention and that this logic may be implemented in either hardware or software. With reference to FIG. 6, the invention is depicted as a software application residing on a personal computer. This is broadly representative of all software implementations including laptop computers, palm top devices, cell phones, calculators, etc.

Instructive Method:

Within the context of the present invention, the accompanying instructive method is provided:
Step 1: The human subject is instructed to position themselves comfortably and to relax for a few moments.
Step 2: The subject is instructed to set the device to 0.085 Hertz.
Step 3: If audible or sensory feedback is desired, the subject is instructed to select these settings and attach any external device such as headphones.
Step 4: If the subject is utilizing the visual capabilities, the subject is instructed to place the invention within plain view.
Step 5: The subject is instructed to turn on the device.
Step 6: The subject is instructed to inhale as the visual display moves upward and exhale as the visual display moves downward. Furthermore, the subject is instructed to time their breathing so as to end inhalation and begin exhalation when the visual display reaches its uppermost point and end exhalation and begin inhalation when the visual display reaches its lowermost point.
Step 7: If the subject is using audible or sensory indicators, they are similarly instructed to align their breathing cycle with audible or sensory cues.
Step 8: The subject is instructed to continue for a specified period of time or as long as is comfortable.
Step 9: If the subject finds their cycle of breathing tends to be faster or slower than 0.085 Hertz (11.8 seconds), they are instructed to increase or decrease the frequency of the device by changing settings on the Setting Selector.
Step 10: The subject is instructed to utilize the device regularly until they have cultivated a clear sense of the rhythm and can employ it at any time.
Step 11: Henceforth, the subject is instructed to utilize the device regularly to maintain and reinforce their ability to synchronize their heart rate variability cycle with their breathing cycle.

1. The broad method of achieving coherence of the heart rate variability cycle by facilitating the synchronization of the heart rate variability cycle with the breathing cycle:
a) The broad method of consciously using the breathing cycle to synchronize the heart rate variability cycle, thereby realizing coherence of said heart rate variability cycle,
b) the broad method of bringing about the coherence of the heart rate variability cycle by consciously aligning the breathing cycle with an external reference signal of a frequency or frequencies known to be consistent with those of the human heart rate variability cycle.

2. The broad system of claim 1 for achieving coherence of the heart rate variability cycle by facilitating the synchronization of the heart rate variability cycle with the breathing cycle:
a. the broad system by which the breathing cycle is used to synchronize the heart rate variability cycle,
b. the broad system by which the coherence of the heart rate variability cycle is achieved by consciously aligning the breathing cycle with an external reference signal of a frequency or frequencies known to be consistent with those of the human heart rate variability cycle.

3. The method of claim 1, further comprising the provision of a consistent cyclic representation of the breathing cycle such that the subject has a clear understanding of when to begin the inhalation phase, when to end the inhalation phase, when to begin the exhalation phase, and when to end the exhalation phase.

4. The system of claim 2, further comprising the provision of a consistent cyclic representation of the breathing cycle such that the subject has a clear understanding of when to begin the inhalation phase, when to end the inhalation phase, when to begin the exhalation phase, and when to end the exhalation phase.

5. The method of claim 1, further comprising providing the subject with an exacting visual, audible, or sensory reference signal specifying the inhalation phase and the exhalation phase.

6. The system of claim 2, further comprising providing the subject with an exacting visual, audible, or sensory reference signal specifying the inhalation phase and the exhalation phase.

7. The method of claim 1 further comprising the synchronization of the peak positive heart rate with the visual, audible, and sensory positive peaks, and the synchronization of the peak negative heart rate with visual, audible, and sensory negative peaks.

8. The system of claim 2, further comprising the synchronization of the peak positive heart rate with the visual, audible, and sensory positive peaks, and the synchronization of the peak negative heart rate with visual, audible, and sensory negative peaks.

9. The method of claim 1, wherein a consistent cyclic representation of the breathing cycle is provided visually, audibly, and sensorally.

10. The system of claim 2, wherein a consistent cyclic representation of the breathing cycle is provided visually, audibly, and sensorally.

11. The method of claim 3 wherein an indication of short duration is alternatively provided at the end of inhalation and the beginning of exhalation and at the end of exhalation and the beginning of exhalation.

12. The system of claim 4 wherein an indication of short duration is alternatively provided at the end of inhalation and the beginning of exhalation and at the end of exhalation and the beginning of inhalation.

13. The method of claim 1 comprising the programmability of differing frequencies centered around 0.085 Hertz.

14. The system of claim 1 comprising the programmability of differing frequencies centered around 0.085 Hertz.

15. The method of claim 5 comprising the programmability of differing indication methods individually or in combination.

16. The system of claim 6 comprising the programmability of differing indication methods individually or in combination.

17. The method of claim 1, wherein the capability may be provided in either hardware or software.

18. The system of claim 1, wherein the capability may be provided in either hardware or software.

19. The instructive method by which the subject is instructed to synchronize their breathing cycle to that of an external reference signal centered around 0.085 Hertz.

20. The instructive method by which the subject is instructed to synchronize their inhalation with the positive going aspect of the external reference cycle and their exhalation with the negative going aspect of the external reference cycle.
